(19) United States
(12) Patent Application Publication
(10) Pub. No.: US 2026/0130346 A1
(43) Pub. Date: May 14, 2026

(54) Method of Erasing Abnormal Epigenetic Modification in Ovine iCHI Embryo and Use in Generating Gene-Edited Sheep

(71) Applicant: Inner Mongolia University, Huhhot (CN)

(72) Inventors: Lei YANG, Huhhot (CN); Anqi DI, Huhhot (CN); Lishuang SONG, Huhhot (CN); Xuefei LIU, Huhhot (CN); Di WU, Huhhot (CN); Song WANG, Huhhot (CN); Zhenting HAO, Huhhot (CN); Lige BU, Huhhot (CN); Chunling BAI, Huhhot (CN); Guanghua SU, Huhhot (CN); Zhuying WEI, Huhhot (CN); Li ZHANG, Huhhot (CN); Zhonghua LIU, Huhhot (CN); Shaorong GAO, Huhhot (CN); Guangpeng LI, Huhhot (CN)

(21) Appl. No.: 19/275,663

(22) Filed: Jul. 21, 2025

(30) Foreign Application Priority Data
Nov. 14, 2024 (CN) 202411620324.5

(51) Int. Cl. A01K 67/0275; C07K 14/47; C12N 5/00; C12N 5/073; C12N 9/22; C12N 15/11; C12N 15/64; C12N 15/66; C12N 15/85; C12N 15/90

(52) U.S. Cl. CPC A01K 67/0275; C07K 14/4716; C12N 5/0018; C12N 5/0604; C12N 9/226; C12N 15/111; C12N 15/64; C12N 15/66; C12N 15/8509; C12N 15/907; A01K 2217/07; A01K 2227/103; C12N 2310/20; C12N 2501/115; C12N 2501/16; C12N 2501/415; C12N 2501/727; C12N 2501/999; C12N 2506/02

(57) ABSTRACT

A method of erasing an abnormal epigenetic modification in a sheep iCHI embryo and use thereof. A method including the steps of: transferring a recombinant plasmid expressing protamine into a sheep androgenetic haploid embryonic stem cell, then introducing the cell into a mature sheep oocyte, and performing activation and culture to obtain a sheep iCHI embryo without abnormal epigenetic modifications. In the present disclosure, abnormal methylations in iCHI embryos may be erased by transient expression of protamine in o-haSCs. The blastocyst rate of the prepared Pro-iCHI embryos is significantly higher than that of iCHI embryos, and is similar to that of IVF embryos. The resulting embryos can be used for embryo transfer to obtain semi-cloned gene-edited sheep, providing materials for subsequent breeding applications.

### CROSS-REFERENCE TO RELATED APPLICATION

This patent application claims the benefit and priority of Chinese Patent Application No. 202411620324.5 filed with the China National Intellectual Property Administration on Nov. 14, 2024, the disclosure of which is incorporated by reference herein in its entirety as part of the present application.

### REFERENCE TO SEQUENCE LISTING

A computer readable XML file entitled “GWP20241208217_seqlist”, which was created on Jul. 3, 2025, with a file size of about 15,321 bytes, contains the sequence listing for this application, has been filed with this application, and is hereby incorporated by reference in its entirety.

### TECHNICAL FIELD

The present disclosure relates to the technical field of genetic engineering, and in particular to a method of erasing an abnormal epigenetic modification in a sheep iCHI embryo and the use thereof.

### BACKGROUND

Haploid androgenetic stem cells (haSCs), also known as “artificial spermatids” can be used for sperm research and have solved the problem of culturing sperm in vitro. haSC-mediated semi-cloning technology has wide application prospects, allowing for the rapid preparation of gene-edited animals, and enabling the genetic screening at the individual level and studies of molecular mechanisms.

Semi-cloned sheep can be prepared by the oocyte intracytoplasmic haSCs injection (iCHI) technique, and ovine haSCs (o-haSCs) used to prepare iCHI embryos can also be subjected to gene editing to obtain gene-edited semi-cloned animals. Like in vitro fertilized embryos, iCHI embryos can form pseudo-pronucleus and undergo DNA demethylation. However, iCHI embryos during development face the same problem as those prepared by somatic cell nuclear transfer (SCNT), both of which show abnormal epigenetic modifications, resulting in the failure of development of reconstituted embryos to blastocysts in vitro. Therefore, erasing incorrect epigenetic modifications to obtain iCHI embryos is crucial for generating gene-edited animals.

### SUMMARY

To address the above problem, the present disclosure provides a method of erasing an abnormal epigenetic modification in a sheep iCHI embryo and use thereof. In the present disclosure, abnormal epigenetic modifications (abnormal methylations) in iCHI embryos can be erased by transient expression of protamine in o-haSCs, solving the problem that sheep iCHI embryos cannot develop to blastocysts.

To achieve the objective described above, the present disclosure provides the following technical solutions.

The present disclosure provides a method of erasing an abnormal epigenetic modification in a sheep iCHI embryo, which includes the steps of:
transferring a recombinant plasmid expressing protamine into an ovine haploid androgenetic stem cells (o-haSCs) to obtain an episomal-Prm1 transient transfection positive cells;
introducing the episomal-Prm1 transient transfection positive cells into a mature oocyte to obtain a Pro-iCHI embryo; and
activating the Pro-iCHI embryo using ionomycin and performing culture to obtain an iCHI embryo in which the abnormal epigenetic modification is erased.

In some embodiments, a method of constructing the recombinant plasmid includes:
subjecting a pCW57-MCS1-P2A-MCS2-mDux vector as a backbone to double digestion using NheI and MluI to obtain a vector fragment of 7,847 bp in size;
synthesizing a recombinant fragment containing Prm1 gene, where the recombinant fragment has the nucleotide sequence of SEQ ID NO: 1;
ligating the vector fragment and the recombinant fragment via DNA ligase to obtain a Prm1 plasmid;
subjecting the Prm1 plasmid to digestion at a NotI restriction site to obtain a linearized Prm1 plasmid;
subjecting a pCRISPR-S12 vector to PCR amplification using a primer episomal-F and a primer episomal-R to obtain a PCR product, where the primer episomal-F and the primer episomal-R have the nucleotide sequences of SEQ ID NO: 2 and SEQ ID NO: 3, respectively; and
subjecting the linearized Prm1 plasmid and the PCR product to homologous recombination to obtain the recombined plasmid.

In some embodiments, a method of the culture includes: culturing the activated Pro-iCHI embryo in a G1 medium for 3 days, and then in a G2 medium for 4 days.

In some embodiments, a sheep haploid androgenetic blastocyst is prepared, and the o-haSCs are cultured; preparation of the sheep haploid androgenetic blastocyst includes a first method or a second method;
the first method includes: removing spindle from a mature sheep oocyte and injecting a sheep sperm into the cytoplasm to obtain a reconstituted embryo; activating the reconstituted embryo in ionomycin, and then performing culture in a medium containing 6-dimethylaminopurine to obtain a blastocyst, i.e., the sheep haploid androgenetic blastocyst;
the second method includes: removing female pronucleus from a fertilized sheep egg, and performing culture in a G1 medium to obtain a cleaved embryo; culturing the cleaved embryo in a G2 medium to obtain a blastocyst, i.e., the sheep haploid androgenetic blastocyst;
a method of culturing the o-haSC includes: after removing zona pellucida, culturing the sheep haploid androgenetic blastocyst in FACE medium to obtain the o-haSC; the FACE medium includes mTeSR1 as a basal medium, and further includes components at the following concentrations: 12 to 15 ng/mL fibroblast growth factor 2, 15 to 20 ng/mL activin A, 6 to 10 μM CHIR99021 and 4 to 8 μM IWR1-1-endo.

In some embodiments, in the first method, the ionomycin has a concentration of 5 to 10 μM, the activating is performed for 5 min, and the 6-dimethylaminopurine in the medium has a concentration of 2 mM.

In some embodiments, the ionomycin has a concentration of 5 μM, and the activating is performed for 5 to 10 min.

The present disclosure provides use of the method of the above technical solution in the preparation of a gene-edited sheep.

The present disclosure provides a method of preparing a gene-edited sheep, which includes the steps of:
introducing a gene editing reagent into o-haSCs to obtain gene-edited o-haSCs;
treating the gene-edited o-haSCs using the method of the above technical solution to obtain a gene-edited sheep iCHI embryo; and
transferring the gene-edited sheep iCHI embryo into a recipient ewe in heat to obtain the gene-edited sheep.

In some embodiments, the gene editing reagent includes a reagent that edits MSTN gene.

In some embodiments, the reagent that edits MSTN gene includes an episomal plasmid-based prime editor (ePE) plasmid; a method of constructing the ePE plasmid includes:
subjecting a pCMV-PE2-P2A-GFP plasmid to double digestion using SgrDI and Pmel to obtain a plasmid fragment of 8,103 bp;
subjecting bGHpoly-gRNA scaffold and the plasmid fragment to homologous recombination to obtain a recombinant fragment, where the bGHpoly-gRNA scaffold has the nucleotide sequence of SEQ ID NO: 4;
subjecting a pCRISPR-S12 plasmid to double digestion using BamHI and PacI to obtain a pCRISPR-S12 plasmid fragment;
subjecting the recombinant fragment and the pCRISPR-S12 plasmid fragment to homologous recombination to obtain an ePE backbone plasmid;
subjecting the ePE backbone plasmid to double digestion using NheI and HindIII, and performing recovery to obtain a linearized ePE backbone plasmid of 18,969 bp in size; and
subjecting the linearized ePE backbone plasmid and pegRNA to homologous recombination to obtain the ePE plasmid, where the pegRNA has the nucleotide sequence of SEQ ID NO: 5.

### Beneficial Effects

The present disclosure provides a method of erasing an abnormal epigenetic modification in a sheep iCHI embryo, which includes the steps of: transferring a recombinant plasmid expressing protamine into o-haSCs to obtain an episomal-Prm1 transient transfection positive cells; introducing the episomal-Prm1 transient transfection positive cells into a mature sheep oocyte to obtain a Pro-iCHI embryo; and activating the Pro-iCHI embryo using ionomycin, and performing culture to obtain the sheep iCHI embryo in which the abnormal epigenetic modification is erased. In the present disclosure, abnormal methylations in iCHI embryos can be erased by transient expression of protamine in o-haSCs. The blastocyst rate of the prepared Pro-iCHI embryos is significantly higher than that of iCHI embryos, and is similar to that of in vitro fertilization (IVF) embryos, providing materials for subsequent breeding applications. The resulting embryos may be used for embryo transfer to generate semi-cloned gene-edited sheep.

### BRIEF DESCRIPTION OF THE DRAWINGS

To describe the technical solutions in the examples of the present disclosure or in the prior art more clearly, the accompanying drawings required for the examples will be briefly described below.

FIGS. 1A-1B are schematic diagrams of iCHI embryo production;

FIGS. 2A-2B show blastocyst rates of the embryos obtained by different methods of iCHI, Pro-iCHI and IVF before implantation;

FIG. 3 shows the immunostaining for H3K4me3, H3K9me3 and H3K27me3 in o-haSCs;

FIG. 4 is a schematic diagram showing the MSTN editing strategy for preparing MSTN gene-edited sheep using Pro-iCHI;

FIG. 5 shows Sanger sequencing results of target sites in MSTN gene-edited sheep and wild-type MSTN gene (SEQ ID NOS: 11 and 12);

FIG. 6 shows results of Western blot analysis on MSTN gene of MSTN gene-edited sheep and wild-type;

FIG. 7 is a physical image of MSTN gene-edited sheep.

### DETAILED DESCRIPTION OF THE EMBODIMENTS

The present disclosure provides a method of erasing an abnormal epigenetic modification in a sheep iCHI embryo, which includes the steps of:
transferring a recombinant plasmid expressing protamine into o-haSCs to obtain an episomal-Prm1 transient transfection positive cells;
introducing the episomal-Prm1 transient transfection positive cells into a mature sheep oocyte to obtain a Pro-iCHI embryo; and
activating the Pro-iCHI embryo using ionomycin, and performing culture to obtain the sheep iCHI embryo in which the abnormal epigenetic modification is erased.

The method provided by the present disclosure is an improved iCHI technique, which repairs abnormal DNA methylations in iCHI embryos during development by transient expression of protamine in o-haSCs, so that the embryos can develop to blastocysts for use in the subsequent production of semi-cloned animals.

As an embodiment, the method of preparing the recombinant plasmid includes the steps of:
subjecting a pCW57-MCS1-P2A-MCS2-mDux vector as a backbone to double digestion using NheI and MluI to obtain a vector fragment of 7,847 bp in size;
synthesizing a recombinant fragment containing Prm1 gene, where the recombinant fragment has a nucleotide sequence as shown in SEQ ID NO: 1;
linking the vector fragment and the recombinant fragment via DNA ligase to obtain a Prm1 plasmid;
subjecting the Prm1 plasmid to digestion at a NotI restriction site to obtain a linearized Prm1 plasmid;
subjecting a pCRISPR-S12 vector to PCR amplification using a primer episomal-F and a primer episomal-R to obtain a PCR product, where the primer episomal-F and the primer episomal-R have nucleotide sequences as shown in SEQ ID NO: 2 and SEQ ID NO: 3, respectively; and
subjecting the linearized Prm1 plasmid and the PCR product to homologous recombination to obtain the recombined plasmid.

As an embodiment, a method of the culture includes: culturing the activated Pro-iCHI embryo in a G1 medium for 3 days, and then in a G2 medium for 4 days.

As an embodiment, a sheep haploid androgenetic blastocyst is prepared, and the o-haSCs are cultured; the method of preparing a sheep haploid androgenetic blastocyst includes a first method or a second method;
the first method includes: removing spindle from a mature sheep oocyte and injecting a sheep sperm into the cytoplasm to obtain a reconstituted embryo; activating the reconstituted embryo in ionomycin, and then performing culture in a medium containing 6-dimethylaminopurine to obtain a blastocyst, i.e., the sheep haploid androgenetic blastocyst;
the second method includes: removing female pronucleus from a fertilized egg of sheep, and performing culture in a G1 medium to obtain a cleaved embryo; culturing the cleaved embryo in a G2 medium to obtain a blastocyst, i.e., the sheep haploid androgenetic blastocyst;
the method of culturing o-haSCs includes: after removing zona pellucida, culturing the sheep haploid androgenetic blastocyst in FACE medium to obtain the o-haSCs; the FACE medium includes mTeSR1 as a basal medium, and further includes components at the following concentrations: 12 to 15 ng/mL fibroblast growth factor 2, 15 to 20 ng/mL activin A, 6 to 10 μM CHIR99021 and 4 to 8 μM IWR1-1-endo.

As another embodiment, the o-haSCs obtained using the FACE medium are sorted by flow cytometry every 5 passages to maintain the proportion of haploid embryonic stem cells.

As an embodiment, in the first method, the concentration of the ionomycin is 5 to 10 μM, the time of the activating is 5 to 10 min, and the concentration of the 6-dimethylaminopurine in the medium is 2 mM.

As an embodiment, the concentration of the ionomycin is 5 μM, and the time of the activating is 5 to 10 min.

On the basis of the above advantages, the present disclosure provides use of the method of the above technical solution in the preparation of a gene-edited sheep.

The present disclosure provides a method of preparing a gene-edited sheep, which method includes the steps of:
introducing a gene editing reagent into o-haSCs to obtain gene-edited o-haSCs;
treating the gene-edited o-haSCs using the method of the above technical solution to obtain a gene-edited sheep iCHI embryo; and
transferring the gene-edited sheep iCHI embryo into a recipient ewe in heat to obtain the gene-edited sheep.

As an embodiment, the gene editing reagent includes a reagent that edits MSTN gene.

As an embodiment, the reagent that edits MSTN gene includes an ePE plasmid; a method of constructing the ePE plasmid includes:
subjecting a pCMV-PE2-P2A-GFP plasmid to double digestion using SgrDI and Pmel to obtain a plasmid fragment of 8,103 bp;
subjecting bGHpoly-gRNA scaffold and the plasmid fragment to homologous recombination to obtain a recombinant fragment, where the bGHpoly-gRNA scaffold has the nucleotide sequence of SEQ ID NO: 4;
subjecting a pCRISPR-S12 plasmid to double digestion using BamHI and PacI to obtain a pCRISPR-S12 plasmid fragment;
subjecting the recombinant fragment and the pCRISPR-S12 plasmid fragment to homologous recombination to obtain an ePE backbone plasmid;
subjecting the ePE backbone plasmid to double digestion using NheI and HindIII, and performing recovery to obtain a linearized ePE backbone plasmid of 18,969 bp in size; and
subjecting the linearized ePE backbone plasmid and pegRNA to homologous recombination to obtain the ePE plasmid, where the pegRNA has the nucleotide sequence of SEQ ID NO: 5.

The ePE plasmid provided by the present disclosure can edit sheep MSTN gene to generate a single base mutation, i.e., G>A, in the 3′UTR region of the MSTN gene, finally obtaining a 14-week preterm MSTN gene-edited sheep.

To further illustrate the present disclosure, the method of erasing an abnormal epigenetic modification in a sheep iCHI embryo and use thereof provided by the present disclosure will be described in detail in conjunction with the accompanying drawings and examples below, which cannot be construed as limiting the protection scope of the present disclosure.

### Example 1

The method of constructing a non-genomically integrated protamine-containing plasmid (episomal-Prm1) was performed as follows.

A pCW57-MCS1-P2A-MCS2-mDux vector (catalog number: 138320) purchased from addgene website, used as a backbone, was double-digested using NheI and MluI to obtain two fragments of 2,184 bp and 7,847 bp in size. The fragment of 7,847 bp in size was recovered using an agarose gel DNA recovery kit (TIANGEN®) to obtain the vector fragment. A recombinant fragment of the murine Prm1 gene and enhanced green fluorescent protein (EGFP) (SEQ ID NO: 1), which had NheI and MluI restriction sites at both ends, was synthesized by Sangon Biotech (Shanghai) Co., Ltd., and the vector fragment and the recombinant fragment were linked via DNA ligase to obtain the Prm1 plasmid.

The Prm1 plasmid was subjected to single digestion at the NotI restriction site to obtain the linearized Prm1 plasmid.

The pCRISPR-S12 vector (addgene, catalog number: 084031) was subjected to PCR amplification using primers episomal-F (SEQ ID NO: 2) and episomal-R (SEQ ID NO: 3), resulting in a PCR product of 4,047 bp in size.

The linearized Prm1 plasmid and the PCR product were subjected to homologous recombination using a portion of a cloning kit (Vazyme) to obtain the episomal-Prm1 vector in which the episomal vector element allows protamine to play a role and is not integrated into the genome.

Recombinant fragment (SEQ ID NO: 1):
5′-CCAGATACCGATGCTGCCGCAGCAAAAGCAGGAGCAGATGCC
GCCGTCGCAGGCGAAGATGTCGCAGACGGAGGAGGCGATGCTGCC
GGCGGAGGAGGCGAAGATGCTGCCGTCGCCGCCGCTCATACACCA
TAAGGTGTAAAAAATACGCCACCATGGTGAGCAAGGGCGAGGAGC
TGTTCACCGGGGTGGTGCCCATCCTGGTCGAGCTGGACGGCGACG
TAAACGGCCACAAGTTCAGCGTGTCCGGCGAGGGCGAGGGCGATG
CCACCTACGGCAAGCTGACCCTGAAGTTCATCTGCACCACCGGCA
AGCTGCCCGTGCCCTGGCCCACCCTCGTGACCACCCTGACCTACG
GCGTGCAGTGCTTCAGCCGCTACCCCGACCACATGAAGCAGCACG
ACTTCTTCAAGTCCGCCATGCCCGAAGGCTACGTCCAGGAGCGCA
CCATCTTCTTCAAGGACGACGGCAACTACAAGACCCGCGCCGAGG
TGAAGTTCGAGGGCGACACCCTGGTGAACCGCATCGAGCTGAAGG
GCATCGACTTCAAGGAGGACGGCAACATCCTGGGGCACAAGCTGG
AGTACAACTACAACAGCCACAACGTCTATATCATGGCCGACAAGC
AGAAGAACGGCATCAAGGTGAACTTCAAGATCCGCCACAACATCG
AGGACGGCAGCGTGCAGCTCGCCGACCACTACCAGCAGAACACCC
CCATCGGCGACGGCCCCGTGCTGCTGCCCGACAACCACTACCTGA
GCACCCAGTCCAAGCTGAGCAAAGACCCCAACGAGAAGCGCGATC
ACATGGTCCTGCTGGAGTTCGTGACCGCCGCCGGGATCACTCTCG
GCATGGACGAGCTGTACAAGTAA-3′;
episomal-F (SEQ ID NO: 2):
5′-ACCACCGCACAGCAAAACGGGTAGCATATGCTTC-3′;
episomal-R (SEQ ID NO: 3):
5′-CAGGTCTGAAGATCAATGTCTGACGAGGGGCCAG-3′.

### Example 2

1. The sheep haploid androgenetic blastocysts were obtained as follows. Mature sheep oocytes were stained for 10 min using 5 μg/mL Hoechst 33342. The position of spindle was determined by a brief irradiation with UV light (1 to 2 s), the spindle was removed in an M2 culture medium containing 5 μg/mL cytochalasin B (CB) using a Nikon® inverted microscope with a 37° C. hot stage, and sperms were injected into the cytoplasm to obtain reconstituted embryos. The reconstituted embryos were then activated for 5 min in the presence of 5 μM ionomycin, and then incubated in 2 mM 6-dimethylaminopurine for 5 h at 37° C., 5% CO₂ saturation humidity to obtain the sheep haploid androgenetic blastocysts.
2. The o-haSCs were cultured as follows: The sheep haploid androgenetic blastocyst was subjected to zona pellucida removing in 0.25% pronase, and then cultured in FACE medium at 37° C., 5% CO₂ saturation humidity until stem cell colonies grew out, where the FACE medium included an mTeSR1 medium as a basal medium, and further contained only components at the following concentrations: 4 μM IWR1-1-endo, 12 ng/mL fibroblast growth factor 2 (FGF2), 8 μM CHIR99021, 15 ng/ml activin A.

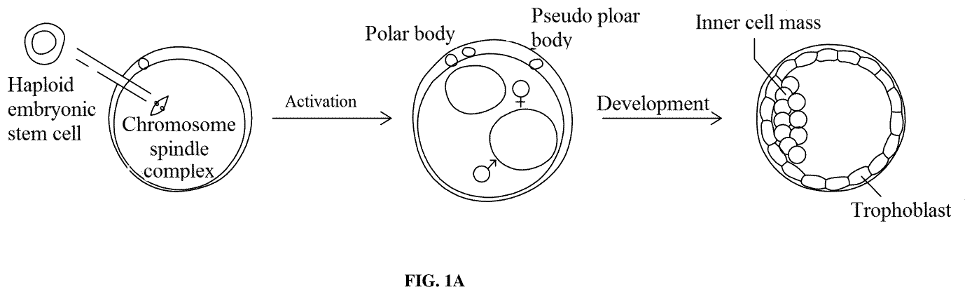

Due to the occurrence of spontaneous diploidization during the culture of sheep androgenetic haploid embryos, and in order to maintain the proportion of haploid cells in the stem cells, the stem cells were continuously sorted by flow cytometry during the culture. The stem cells cultured for 5 passages were incubated for 30 min using 10 μg/mL Hoechst 33342 at 37° C., where the cells were gently and uniformly mixed every 10 min to allow uniform staining. The cell population at n=1 was collected, the spontaneous diploid stem cells were removed, and the stem cells were purified. The results of flow sorting showed that the proportion of sheep androgenetic haploid embryos in the first sorting reached 20%, and with the increase of the frequency of sorting (5 to 6 times), finally the proportion reached 70%.
3. With regard to the o-haSCs after flow sorting in step 2, the medium and the feed layer cells were discarded, and the remaining o-haSCs were then resuspended using FACE medium mixed with 5 μg of episomal-Prm1 vector (constructed in Example 1). The cells were then added to an electroporation cuvette, and electric shock was performed by applying a voltage of 220 V to obtain episomal-Prm1 transient transfection positive o-haSCs.
4. The sheep progenitor iCHI (Pro-iCHI) blastocyst was prepared as follows (see FIG. 1A for a part of the schematic diagram). One episomal-Prm1 transient transfection positive AG-haESC was injected into a mature sheep oocyte in an M2 culture medium containing 5 μg/mL cytochalasin B (CB) under a Nikon® inverted microscope equipped with a 37° C. hot stage to obtain a Pro-iCHI embryo; the Pro-iCHI embryo was buffered in a KSOM-AA medium for 1 h, and then activated for 5 min using 5 μM ionomycin to obtain an activated reconstituted embryo; and the activated reconstituted embryo was cultured in a G1 medium for 3 days, and then cultured in a G2 medium for 4 days to obtain the sheep Pro-iCHI blastocyst that can be used for the preparation of a semi-cloned animal.

### Comparative Example 1

The o-haSCs cultured by the method of steps 1 to 2 of Example 2 were used.

Preparation of sheep iCHI embryo: One o-haSC was injected into a mature sheep oocyte in an M2 culture medium containing 5 μg/mL cytochalasin B (CB) under a Nikon® inverted microscope equipped with a 37° C. hot stage to obtain a reconstituted embryo. The reconstituted embryo was activated for 5 min in the presence of 5 μM ionomycin, and then incubated in 2 mM 6-dimethylaminopurine for 5 h at 37° C., 5% CO₂ saturation humidity to obtain the sheep iCHI embryo. The morphological pictures of the iCHI embryo formed from the sheep androgenetic haploid embryo are shown in FIG. 1B, where PB is a polar body; PPB is a pseudo polar body; the scale bar is 100 μm.

### Test Example 1

Preparation of IVF embryo: in vitro mature sheep oocytes were placed in a fertilization liquid with added sperms, at a sperm-to-oocyte ratio of 500:1. After 6 h of fertilization, the sperms were washed off, culture was performed in a G1 medium for 3 days, and then culture was performed in a G2 medium for 4 days to obtain blastocysts.

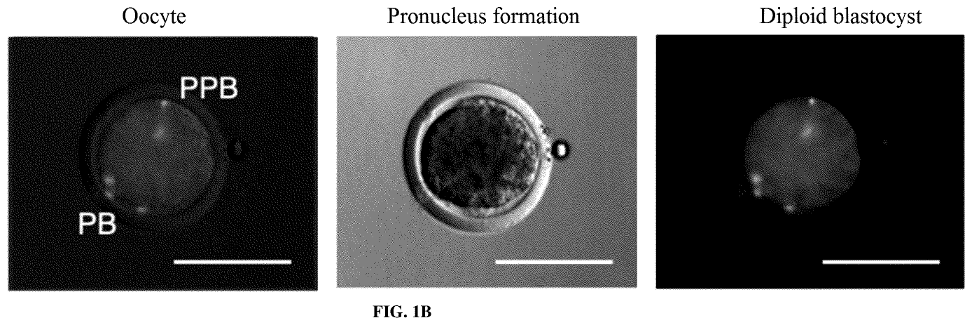

The developments of the sheep Pro-iCHI blastocysts prepared in Example 2, the sheep iCHI embryos prepared in Comparative example 1, and the IVF embryos prepared by the above method were observed and counted, and the experiments were triplicated. The results are shown in FIGS. 2A-2B and Table 1.

TABLE 1
Development of iCHI and Pro-iCHI embryos before implantation

| | Number | | | | | |
|---|---|---|---|---|---|---|
| | of 1-cell | Cleavage | | | | |
| | embryos | rate of 2-cell | 4-cell | 8-cell | | Blastocyst |
| | (1-cell | embryos | embryo rate | embryo rate | Morula rate | rate |
| Grouping | embryo) | (%) | (%) | (%) | (%) | (%) |
| iCHI | 415 | 61.18 ± 0.31*** | 86.15 ± 2.35 | 77.37 ± 2.40*** | 33.68 ± 2.75*** | 24.22 ± 2.21*** |
| Pro-iCHI | 381 | 75.37 ± 2.05 | 87.68 ± 1.88 | 80.20 ± 3.86 | 62.88 ± 2.29 | 51.22 ± 1.78 |
| IVF | 517 | 77.98 ± 1.38 | 87.41 ± 1.83 | 82.33 ± 1.15 | 62.41 ± 2.50 | 54.03 ± 2.44 |

Note:
***indicates P < 0.001, using two-tailed t-test; Cleavage rate of 2-cell embryos = (number of 2-cell embryos/number of 1-cell embryos) × 100%, The cleavage rates at other developmental stages are calculated as the number of cleaved embryos at each time point divided by the number of 2-cell embryos,, expressed as a percentage.

The results indicated that the blastocyst rate of Pro-iCHI embryos was significantly higher than that of iCHI embryos, and was similar to that of IVF embryos. By the method provided by the present disclosure, abnormal methylations in iCHI embryos can be erased by transient expression of protamine in o-haSCs, so that the Pro-iCHI embryos can develop to blastocysts, providing materials for subsequent breeding applications.

### Test Example 2

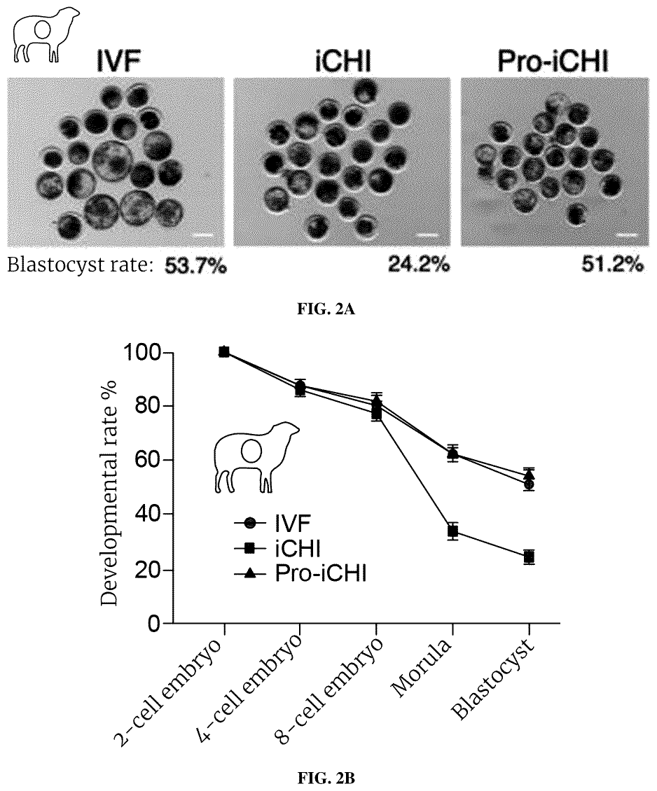

The episomal-Prm1 transient transfection positive cells prepared by the method of Example 2 at different times (0 h, 24 h and 48 h) each were immunostained for histone H3 trimethylated at lysine 4 (H3K4me3), histone H3 trimethylated at lysine 9 (H3K9me3) and histone H3 trimethylated at lysine 27 (H3K27me3). The results are shown in FIG. 3, where the scale bar is 20 μm, 0 h represents the episomal-Prm1 transient transfection positive cells at 0 h after the completion of electroporation, 24 h represents the episomal-Prm1 transient transfection positive cells at 24 h after the completion of electroporation, and 48 h represents the episomal-Prm1 transient transfection positive cells at 48 h after the completion of electroporation.

It could be seen from the results that transiently expressed protamine could erase abnormal modifications, i.e., H3K4me3, H3K9me3 and H3K27me3 in an iCHI embryo, and compressed the nucleus into a sperm-like structure.

### Example 3

In the present disclosure, to better use the Pro-iCHI method, the myostatin (MSTN) gene of o-haSCs was selected for gene editing, and the gene-edited semi-cloned animal was prepared using Pro-iCHI technology. MSTN gene, predominantly expressed in skeletal muscle, is a member of the transforming growth factor-beta (TGF-beta) superfamily, which is expressed in all tissues. If a mutation occurs in MSTN gene, it can lead to significantly developed muscle groups in animal, resulting in a “double-muscling” phenotype. This gene is thus selected as a target site for gene editing in sheep, which can not only improve the commercial value of the sheep, but also allows for observation of obvious traits.

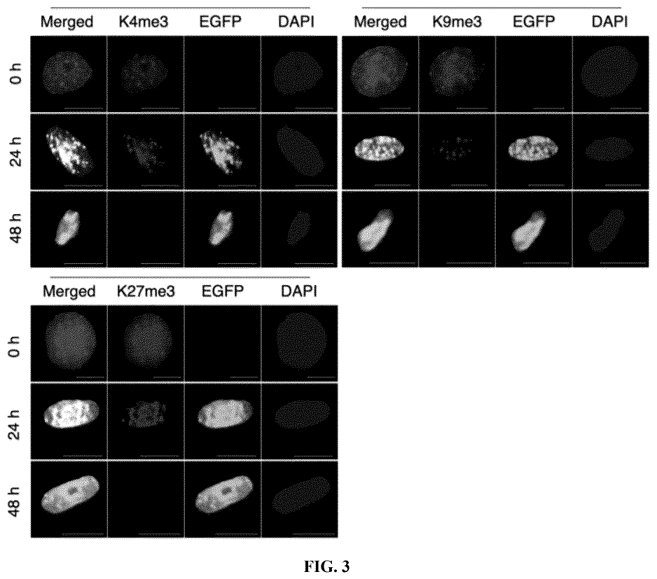

In the present disclosure, prime editor (PE) was selected as a sheep MSTN gene editing tool. The main working principle of the PE is “searching” and “replacing” target sites without introducing double strand breaks and donor DNA templates to achieve the accurate insertion and deletion of multiple bases. The editing principle of the prime editor is shown in FIG. 4: under the guidance of a spacer sequence on the prime editing guide RNA (pegRNA), Cas9 nickase (nCas9) cleaves single-stranded DNA at Nick. The sequence of the primer binding site (PBS) (i.e. primer sequence) recognizes the complementary sequence before the Nick site, and using a reverse transcription template (RTT) sequence as a template for reverse transcription, the sequence of interest is polymerized onto the nicked DNA strand. The process is as follows.
1) The pCMV-PE2-P2A-GFP plasmid (purchased from addgene, catalog number: #132776) was double digested using SgrDI and Pmel. The fragments of interest were verified by DNA gel electrophoresis, and the fragment of 8,103 bp in length was recovered using a gel recovery kit (TIANGEN), resulting in a linearized plasmid fragment.
2) The bGHpoly-gRNA scaffold sequence fragment was synthesized by Sangon Biotech (Shanghai) Co., Ltd., and integrated with the linearized plasmid fragment recovered in step 1) using ClonExpress Ultra one step cloning kit V2 (Vazyme) to obtain an integrated fragment. The bGHpoly-gRNA scaffold sequence fragment has the nucleotide sequence of SEQ ID NO: 4, specifically as follows: 5′-ctgtgccttctagttgccagccatctgttgtttgcccctcccccgtgccttccttgaccctggaaggtgccactcccactgtcctttcctaata aaatgaggaaattgcatcgcattgtctgagtaggtgtcattctattctggggggtggggggggcaggacagcaagggggaggattggga agagaatagcaggcatgctggggagcggccgcgcctttttacggttcctggccttttgctggccttttgctcacatgtgagggcctatttccca tgattccttcatatttgcatatacgatacaaggctgttagagagataattggaattaatttgactgtaaacacaaagatattagtacaaaatacgtg acgtagaaagtaataatttcttgggtagtttgcagttttaaaattatgttttaaaatggactatcatatgcttaccgtaacttgaaagtatttcgatttct tggctttatatatcttgtggaaaggacGCTAGCgaaacaccgattttgttttgatgtattcaCTCGAGgttttagagctagaaatagca agttaaaataaggctagtccgttatcaacttgaaaaagtggcaccgagtcggtgc-3′.
3) The pCRISPR-S12 vector (addgene, catalog number: Plasmid 084031) was subjected to double digestion using BamHI and PacI, and the resulting vector and the integrated fragment obtained in step 2) were subjected to homologous recombination to obtain an ePE backbone plasmid; the ePE backbone plasmid was subjected to double digestion of NheI and HindIII and recovery to obtain a linearized ePE backbone plasmid of 18,969 bp in size. The pegRNA (SEQ ID NO: 5) was artificially synthesized, and homologously recombined with the linearized ePE backbone plasmid, resulting in an ePE plasmid for use in the subsequent editing of sheep MSTN gene.

SEQ ID NO: 5:
5′-ATTTATAAGTATTAAAATAAgttttagagctagaaatagcaa
gttaaaataaggctagtccgttatcaacttgaaaaagtggcaccg
agtcggtgcAACATTCCATTATTTTAATACTTATAAAT-3′;
where,
5′-ATTTATAAGTATTAAAATAA-3′
(SEQ ID. 6) is a spacer sequence,
5′-gttttagagctagaaatagcaagttaaaataaggctag
tccgttatcaacttgaaaaagtggcaccgagtcggtgc-3′
(SEQ ID NO: 7)

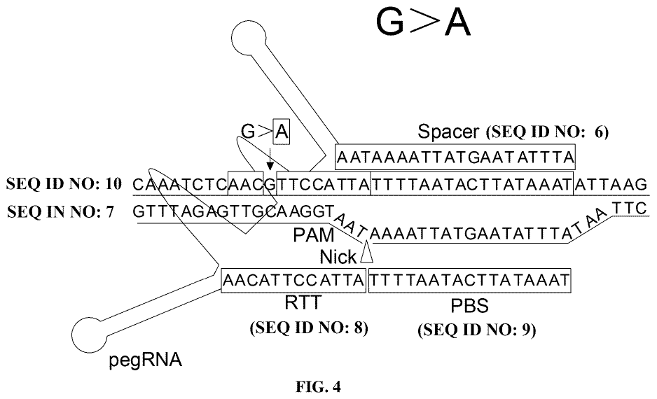

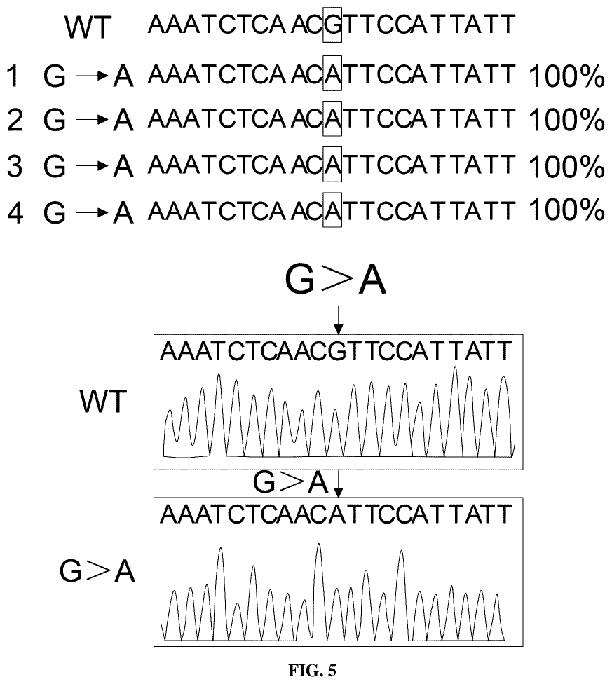

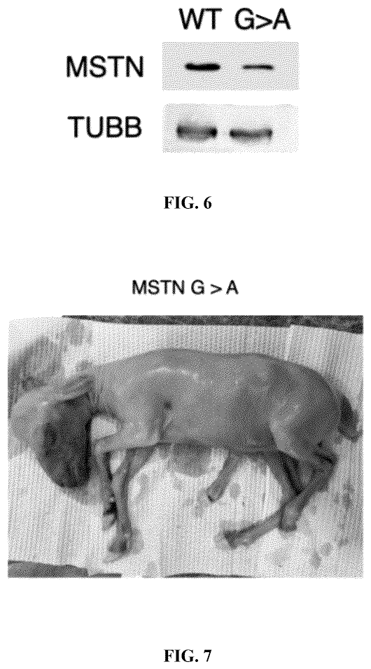

is a gRNA scaffold, 5′-AACATTCCATTA-3′ (SEQ ID NO: 8) is the RTT sequence, and 5′-TTTTAATACTTATAAAT-3′ (SEQ ID NO: 9) is the PBS sequence.
4) The o-haSCs, which were cultured using the method of steps 1 to 2 of Example 2, were used for the introduction of the ePE plasmid obtained in step 3). The results of gene editing were detected using dideoxy sequencing (Sanger sequencing) and Western Blot. The results of Sanger sequencing are shown in FIG. 5. The detection results of Western Blot are shown in FIG. 6. It was observed that a single base mutation, i.e., G>A, was generated in the genome of MSTN, and the expression of MSTN protein was also lower than that in unedited cells (wild-type, denoted as WT), resulting in the generation of the o-haSCs with MSTN knockout.
5) The o-haSCs with MSTN knockout obtained in step 4) were resuspended using FACE medium mixed with 5 μg of episomal-Prm1 vector (constructed in Example 1). The cells were then added to an electroporation cuvette, and electric shock was performed by applying a voltage of 220 V to obtain episomal-Prm1 transient transfection positive o-haSCs with MSTN knockout.
6) One episomal-Prm1 transient transfection positive o-haSCs with MSTN knockout obtained in step 5) was injected into a mature sheep oocyte in an M2 culture medium containing 5 g/mL cytochalasin B (CB) under a Nikon® inverted microscope equipped with a 37° C. hot stage to obtain a Pro-iCHI embryo with MSTN knockout; the Pro-iCHI embryo with MSTN knockout was buffered in a KSOM-AA medium for 1 h, and then activated for 5 min using 5 μM ionomycin to obtain an activated reconstituted embryo; and the activated reconstituted embryo was cultured in a G1 medium for 3 days, and then cultured in a G2 medium for 4 days to obtain a sheep Pro-iCHI blastocyst with MSTN knockout for use in subsequent embryo transfer.
7) Estrus synchronization treatment was performed on recipient ewes using vaginal suppository, cloprostenol and gonadotropin releasing hormone. The blastocyst with MSTN knockout obtained in step 6) was stored in an M199 medium (Thermo) containing 15% (V/V) fetal bovine serum, and surgically transferred into a recipient sheep using a TomCat catheter (Sovereign*). In the present disclosure, a total of 256 2-cell embryos were transferred into 32 recipient sheep, with 8 embryos per sheep, and finally a 14-week preterm MSTN gene-edited sheep was obtained (see FIG. 7 and Table 2).

TABLE 2
Transfer and production of sheep Pro-iCHI embryos

| | | | Cleavage rate | | | | | Number of | |
|---|---|---|---|---|---|---|---|---|---|
| | | | of 2-cell | | | Number of | | litters born | |
| | | Number of | embryos (% | | Embryo | transferred | Number | (% of | |
| | MSTN | reconstituted | of 1-cell | | transfer | embryos per | of | transferred | |
| Edit type | site | embryos | embryos) | | stage | recipient | recipients | embryos) | |
| / | Wild- | 122 | 92 | (75.40) | 2-cell | 8 | 9 | 0 | (0) |
| | type | | | | embryo | | | | |
| Mutation | G > A | 362 | 272 | (75.14) | 2-cell | 8 | 32 | 1 | (0.39) |
| | | | | | embryo | | | | |

Note:
The wild-type in Table 2 represents sheep Pro-iCHI blastocysts in which MSTN gene is not edited using the ePE plasmid.

### Example 4

The method was similar to that of Example 2, except that the method of obtaining the sheep haploid androgenetic blastocysts in step 1. Specifically, the fertilized eggs were first obtained by in vitro fertilization, and stained with 5 μg/mL Hoechst 33342 for 10 min in a G1 medium (Vitrolife). The positions of male and female pronuclei were determined by a brief irradiation with UV light (1 to 2 s). The female pronucleus was then removed by micromanipulation. The fertilized eggs were subsequently cultured in a G1 medium (Vitrolife) for 72 h, then transferred to a G2 medium (Vitrolife), and cultured at 37° C., 5% CO₂ saturation humidity until blastocyst formation, resulting in the production of sheep haploid androgenetic blastocysts.

The blastocyst rates of the haploid androgenetic embryos obtained by the two methods of Example 4 and Example 2 were similar, 25.68% and 25.36%, respectively.

In summary, the method provided by the present disclosure can erase abnormal methylations in iCHI embryos by transient expression of protamine in o-haSCs; the blastocyst rate of the prepared Pro-iCHI embryos is significantly higher than that of iCHI embryos, and is similar to that of IVF embryos; the method also allows the development of Pro-iCHI embryos to blastocysts, providing material for subsequent breeding applications. The resulting embryos can be used for embryo transfer to obtain semi-cloned gene-edited sheep.

Although the examples described above have provided a detailed description of the present disclosure, they are only some, rather than all embodiments of the present disclosure. All other embodiments that can be obtained according to the examples of the present disclosure without involving any inventive effort shall fall within the protection scope of the present disclosure.

What is claimed is:

1. A method of erasing an abnormal epigenetic modification in a sheep iCHI embryo, comprising the steps of:
transferring a recombinant plasmid expressing protamine into ovine haploid androgenetic stem cells (o-haSCs) to obtain episomal-Prm1 transient transfection positive cells;
introducing the episomal-Prm1 transient transfection positive cells into a mature sheep oocyte to obtain a Pro-iCHI embryo; and
activating the Pro-iCHI embryo using ionomycin, and performing culture to obtain an iCHI embryo in which the abnormal epigenetic modification is erased.

2. The method of claim 1, wherein construction of the recombinant plasmid comprises:
subjecting a pCW57-MCS1-P2A-MCS2-mDux vector as a backbone to double digestion using NheI and MluI to obtain a vector fragment of 7,847 bp in size;
synthesizing a recombinant fragment containing Prm1 gene, wherein the recombinant fragment has the nucleotide sequence of SEQ ID NO: 1;
ligating the vector fragment and the recombinant fragment via DNA ligase to obtain a Prm1 plasmid;
subjecting the Prm1 plasmid to digestion at a NotI restriction site to obtain a linearized Prm1 plasmid;
subjecting a pCRISPR-S12 vector to PCR amplification using a primer episomal-F and a primer episomal-R to obtain a PCR product, wherein the primer episomal-F and the primer episomal-R have the nucleotide sequences of SEQ ID NO: 2 and SEQ ID NO: 3, respectively; and
subjecting the linearized Prm1 plasmid and the PCR product to homologous recombination to obtain the recombined plasmid.

3. The method of claim 1, wherein the culture comprises: culturing the activated Pro-iCHI embryo in a G1 medium for 3 days, and then in a G2 medium for 4 days.

4. The method of claim 1, wherein preparation of the o-haESCs comprises: preparing a sheep haploid androgenetic blastocyst and culturing the o-haESCs; preparation of the sheep haploid androgenetic blastocyst is conducted by a first method or a second method;
the first method comprises: removing spindle from a mature sheep oocyte and injecting a sheep sperm into the cytoplasm to obtain a reconstituted embryo; activating the reconstituted embryo in ionomycin, and then performing culture in a medium containing 6-dimethylaminopurine to obtain a blastocyst, i.e., the sheep haploid androgenetic blastocyst;
the second method comprises: removing female pronucleus from a fertilized sheep egg, and performing culture in a G1 medium to obtain a cleaved embryo; culturing the cleaved embryo in a G2 medium to obtain a blastocyst, i.e., the sheep haploid androgenetic blastocyst;
a method of culturing the o-haSCs comprises: after removing zona pellucida, culturing the sheep haploid androgenetic blastocyst in FACE medium to obtain the o-haSCs; the FACE medium comprises mTeSR1 as a basal medium, and further comprises components at the following concentrations: 12 to 15 ng/mL fibroblast growth factor 2, 15 to 20 ng/mL activin A, 6 to 10 μM CHIR99021 and 4 to 8 UM IWR1-1-endo.

5. The method of claim 4, wherein in the first method, the ionomycin has a concentration of 5 to 10 UM, the activating performed for 5 min, and the 6-dimethylaminopurine in the medium has a concentration of 2 mM.

6. The method of claim 1, wherein the ionomycin has a concentration of 5 μM, and the activating is performed for 5 to 10 min.

7. A method of preparing a gene-edited sheep, comprising the steps of:
introducing a gene editing reagent into o-haSCs to obtain gene-edited o-haSCs;
treating the gene-edited o-haSCs using the method of claim 1 to obtain a gene-edited sheep iCHI embryo; and
transferring the gene-edited sheep iCHI embryo into a recipient ewe in heat to obtain the gene-edited sheep.

8. The method of claim 7, wherein construction of the recombinant plasmid comprises:
subjecting a pCW57-MCS1-P2A-MCS2-mDux vector as a backbone to double digestion using NheI and MluI to obtain a vector fragment of 7,847 bp in size;
synthesizing a recombinant fragment containing Prm1 gene, wherein the recombinant fragment has the nucleotide sequence of SEQ ID NO: 1;
ligating the vector fragment and the recombinant fragment via DNA ligase to obtain a Prm1 plasmid;
subjecting the Prm1 plasmid to digestion at a NotI restriction site to obtain a linearized Prm1 plasmid;
subjecting a pCRISPR-S12 vector to PCR amplification using a primer episomal-F and a primer episomal-R to obtain a PCR product, wherein the primer episomal-F and the primer episomal-R have the nucleotide sequences of SEQ ID NO: 2 and SEQ ID NO: 3, respectively; and
subjecting the linearized Prm1 plasmid and the PCR product to homologous recombination to obtain the recombined plasmid.

9. The method of claim 7, wherein the culture comprises: culturing the activated Pro-iCHI embryo in a G1 medium for 3 days, and then in a G2 medium for 4 days.

10. The method of claim 7, wherein preparation of the o-haSCs comprises: preparing a sheep haploid androgenetic blastocyst and culturing the o-haSCs; preparation of the sheep haploid androgenetic blastocyst is conducted by a first method or a second method;
the first method comprises: removing spindle from a mature sheep oocyte and injecting a sheep sperm into the cytoplasm to obtain a reconstituted embryo; activating the reconstituted embryo in ionomycin, and then performing culture in a medium containing 6-dimethylaminopurine to obtain a blastocyst, i.e., the sheep haploid androgenetic blastocyst;
the second method comprises: removing female pronucleus from a zygote, and performing culture in a G1 medium to obtain a cleaved embryo; culturing the cleaved embryo in a G2 medium to obtain a blastocyst, i.e., the sheep haploid androgenetic blastocyst;
a method of culturing the o-haSCs comprises: after removing zona pellucida, culturing the sheep haploid androgenetic blastocyst in FACE medium to obtain the o-haSCs; the FACE medium comprises mTeSR1 as a basal medium, and further comprises components at the following concentrations: 12 to 15 ng/mL fibroblast growth factor 2, 15 to 20 ng/mL activin A, 6 to 10 μM CHIR99021 and 4 to 8 μM IWR1-1-endo.

11. The method of claim 7, wherein in the first method, the ionomycin has a concentration of 5 to 10 UM, the activating performed for 5 min, and the 6-dimethylaminopurine in the medium has a concentration of 2 mM.

12. The method of claim 7, wherein the gene editing reagent includes a reagent that edits MSTN gene.

13. The method of claim 8, wherein the reagent that edits MSTN gene includes an ePE plasmid; a method of constructing the ePE plasmid comprises:
subjecting a pCMV-PE2-P2A-GFP plasmid to double digestion using SgrDI and Pmel to obtain a plasmid fragment of 8103 bp;
subjecting bGHpoly-gRNA scaffold and the plasmid fragment to homologous recombination to obtain a recombinant fragment, wherein the bGHpoly-gRNA scaffold has the nucleotide sequence of SEQ ID NO: 4;
subjecting a pCRISPR-S12 plasmid to double digestion using BamHI and PacI to obtain a pCRISPR-S12 plasmid fragment;
subjecting the recombinant fragment and the pCRISPR-S12 plasmid fragment to homologous recombination to obtain an ePE backbone plasmid;
subjecting the ePE backbone plasmid to double digestion using NheI and HindIII, and performing recovery to obtain a linearized ePE backbone plasmid of 18,969 bp in size; and
subjecting the linearized ePE backbone plasmid and prime editing guide RNA (pegRNA) to homologous recombination to obtain the ePE plasmid, wherein the pegRNA has the nucleotide sequence of SEQ ID NO: 5.


SEQUENCE LISTING (12 sequences)

SEQ ID NO: 1 - DNA, 875 bp, synthetic construct
  source 1..875: mol_type = other DNA; note = recombinant fragment containing Prm1 gene
ccagataccgatgctgccgcagcaaaagcaggagcagatgccgccgtcgcaggcgaagatgtcgcagacggaggaggcgatgctgccggcggaggaggcgaagatgctgccgtcgccgccgctcatacaccataaggtgtaaaaaatacgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggatcactctcggcatggacgagctgtacaagtaa

SEQ ID NO: 2 - DNA, 34 bp, synthetic construct
  source 1..34: mol_type = other DNA; note = primer episomal-F
accaccgcacagcaaaacgggtagcatatgcttc

SEQ ID NO: 3 - DNA, 34 bp, synthetic construct
  source 1..34: mol_type = other DNA; note = primer episomal-R
caggtctgaagatcaatgtctgacgaggggccag

SEQ ID NO: 4 - DNA, 620 bp, synthetic construct
  source 1..620: mol_type = other DNA; note = bGHpoly-gRNA scaffold
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcgcctttttacggttcctggccttttgctggccttttgctcacatgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgctagcgaaacaccgattttgttttgatgtattcactcgaggttttagagctagaaatagcaagttaaaataaggctagtccgttatcaacttgaaaaagtggcaccgagtcggtgc

SEQ ID NO: 5 - DNA, 125 bp, synthetic construct
  source 1..125: mol_type = other DNA; note = pegRNA
atttataagtattaaaataagttttagagctagaaatagcaagttaaaataaggctagtccgttatcaacttgaaaaagtggcaccgagtcggtgcaacattccattattttaatacttataaat

SEQ ID NO: 6 - DNA, 20 bp, synthetic construct
  source 1..20: mol_type = other DNA; note = spacer
atttataagtattaaaataa

SEQ ID NO: 7 - DNA, 43 bp, synthetic construct
  source 1..43: mol_type = other DNA; note = fragment of MSTN gene
cttaatatttataagtattaaaataatggaacgttgagatttg

SEQ ID NO: 8 - DNA, 12 bp, synthetic construct
  source 1..12: mol_type = other DNA; note = RTT sequence
aacattccatta

SEQ ID NO: 9 - DNA, 17 bp, synthetic construct
  source 1..17: mol_type = other DNA; note = PBS sequence
ttttaatacttataaat

SEQ ID NO: 10 - DNA, 43 bp, synthetic construct
  source 1..43: mol_type = other DNA; note = complementary sequence of the fragment of MSTN gene
caaatctcaacgttccattattttaatacttataaatattaag

SEQ ID NO: 11 - DNA, 21 bp, synthetic construct
  source 1..21: mol_type = other DNA; note = sequencing results of wild-type MSTN gene
aaatctcaacgttccattatt

SEQ ID NO: 12 - DNA, 21 bp, synthetic construct
  source 1..21: mol_type = other DNA; note = sequencing results of edited MSTN gene
aaatctcaacattccattatt